(19) United States
(12) Patent Application Publication
(10) Pub. No.: US 2003/0131674 A1
(43) Pub. Date: Jul. 17, 2003

(54) Pole testing system

(76) Inventors: Alfred Denis Foley, Auckland (NZ); Jeffry Paul Foley, Auckland (NZ)

Correspondence Address: JACOBSON HOLMAN, PROFESSIONAL LIMITED LIABILITY COMPANY, 400 SEVENTH STREET, N.W., WASHINGTON, DC, 20004, US

(21) Appl. No.: 10/290,467

(22) Filed: Nov. 8, 2002

Related U.S. Application Data
(60) Provisional application No. 60/331,097, filed on Nov. 8, 2001.

(51) Int. Cl. G01N 33/46; G01N 3/40; G01N 29/18

(52) U.S. Cl. 73/866; 73/81; 73/594; 73/598; 702/39

(57) ABSTRACT

A method of testing a wooden structural member provides a quantitive estimate of the residual strength of the member. The method includes determining the presence of anomalies in each of a plurality of intersecting transverse paths through the structural member at a tested cross section of the structural member. A unit cross section of corresponding shape to the tested cross section has a plurality of predefined regions. For each region of the plurality of regions a determination is made that the region is suspect if each of the paths intersecting at the region has indicated an anomaly. A residual stiffness property for the cross section is calculated discounting at least some of the suspect regions. The calculated residual stiffness property may be displayed together with a visual representation of the cross section of the wooden structural member with the suspect regions highlighted.

Associated apparatus is described and claimed including an implementing computer system and a below ground penetration test tool.

### BACKGROUND TO THE INVENTION

1. Field of the Invention

The present invention relates a testing system for wooden structural members. The system is particularly adapted for testing the integrity and residual strength of utility poles or other ground embedded wooden poles, but may also be applied to other structural members or to trees or logs.

2. Summary of the Prior Art

Existing utility pole networks constitute a significant capital investment component for a utility pole networks constitute a significant capital investment component for a utility line network. A significant proportion of existing, new and replacement utility poles are wooden poles.

Over time, environmental degradation reduces the integrity and structural strength of the utility pole. Environmental degradation can include surface degradation axial splitting, termite damage and internal and surface wood rot, the later particularly associated with moisture absorption through the ground embedded end. To ensure continued utility supply it is important to eliminate network failures caused by avoidable utility pole failure. Accordingly utility poles have generally been subject to a regular visual inspection with replacement of suspect poles. For maximised economic returns it is clear that an inspection programme should lead to replacement of all poles whose residual strength is below a threshold limit but which does not reject poles of acceptable residual strength.

A range of testing procedures and devices has been proposed in the prior art.

In U.S. Pat. No. 4726691 a method of determining the residual stiffness of a wooden pole in place is described. The method involves striking the pole to initiate natural vibration of the structure in a band of frequencies that cover at least two of the first five resonant modes of vibration. A transducer measures the vibrational motion of the structure. Resulting signals are analysed and the results are compared with results from a mathematical model to determine the overall stiffness and condition of the structure.

U.S. Pat. No. 4,329,882 describes a kit of tools for taking physical samples of a pole structure at an underground level to determine the below ground integrity of the structure.

U.S. Pat. No. 4,495,518 describes an enhancement to visual inspection which includes viewing the utility pole in the infrared spectrum. This is said to highlight structurally unsound pole regions to an extent that enables a pole to be inspected from a moving vehicle.

U.S. Pat. No. 4,343,179 describes a device for testing the surface hardness or rot depth of a utility pole. The device includes a test needle which is driven into the pole surface by a known actuation force. The depth of penetration of the needle indicates the rot depth or residual hardness of the material.

U.S. Pat. No. 5,105,453 describes a portable gamma ray device for locating wood rot at longitudinal positions along a utility pole, determining whether the pole is healthy or decayed by the variation in gamma ray attenuation.

U.S. Pat. No. 5,804,728 describes a system for testing utility poles using acousto-ultrasonic transmission across a diameter of the pole and determining frequency response and attenuation characteristics of the signal to determine pole integrity.

Our New Zealand Patent 232756 describes a pole testing method. The method includes performing a hardness test on the pole exterior surface using a Pilodyn wood tester. The Pylodin wood tester includes a steel striker pin impacting the wood surface with known energy so that the depth of penetration indicates residual surface hardness. A residual external diameter of the pole is determined by deducting the depth of penetration of the Pylodin striker. The internal integrity of the pole is determined by ultra sound testing. Integrity is tested across an array of paths connecting between circumferentially distributed transmitter or receiver locations. The results for each transmittal path are recorded as either a “go” or “no-go” determination. It is suggested that the “go” path may subsequently be plotted on a circular chart, with regions showing an absence of lines indicating internal anomalies such as rot or cracks. The total estimated area of anomalies may then be used to calculate a residual factor of safety for the pole. While this system provided a field appropriate testing procedure, manual interpretation of the results is a burdensome task and can only provide a rough estimate of the residual sound cross section of the pole.

### SUMMARY OF THE INVENTION

It is therefore an object of the present invention to provide a method of testing a wooden structural member which is an improvement on the prior art systems and devices or which will at least provide the industry with a useful choice.

In a first aspect the present invention consists in a method of testing a wooden structural member to provide a quantitative estimate of the residual strength thereof comprising, in any order:

determining the presence of anomalies in each of a plurality of intersecting transverse paths through said structural member at a cross section of said structural member,

for each region of a plurality of regions, said plurality of regions together making up a unit cross section of a structural member of the shape being tested and each region including a point of intersection of at least two said paths, determining the region as suspect if each of the paths intersecting at said region has indicated an anomaly, and

calculating a residual stiffness property for said cross section, discounting suspect regions.

In a further aspect the invention consists in a method for testing for internal anomalies in a wooden structural member comprising the steps of:

determining the presence of anomalies in each of a plurality of intersecting transverse paths through said structural member at a cross section of said structural member,

for each region of a plurality of regions, said plurality of regions together making up a unit cross section of a structural member of the shape being tested and each region including a point of intersection of at least two said paths, determining the region as suspect if each of the paths intersecting at said region has indicated an anomaly, and

displaying a visual representation of said cross section of said wooden structural member with suspect regions highlighted.

In a still further aspect the present invention consists in apparatus for use in testing a wooden structural member to provide a quantitative estimate of the residual strength thereof comprising:

means for receiving a determination of the presence of anomalies in each of a plurality of intersecting transverse paths through said structural member at a test cross section of said structural member,

processing means for determining for each region of a plurality of regions, said plurality of regions together making up a unit cross section of a structural member of the shape being tested and each region including a point of intersection of at least two said paths, the region as suspect if each of the paths intersecting at said region has indicated an anomaly, and

processing means for calculating a residual stiffness property for said unit cross section, discounting suspect regions.

In a still further aspect the present invention consists in apparatus for use in testing for internal anomalies in a wooden structural member comprising:

means for receiving a determination of the presence of anomalies in each of a plurality of intersecting transverse paths through said structural member at a cross section of said structural member,

processing means for determining for each region of a plurality of regions, said plurality of regions together making up a unit cross section of a structural member of the shape being tested and each region including a point of intersection of at least two said paths, the region as suspect if each of the paths intersecting at said region has indicated an anomaly,

a display, and

means for displaying a visual representation of said cross section of said wooden structural member with suspect regions highlighted.

In a yet further aspect the invention consists in a below ground penetration test tool comprising:

a plate,

a guide tube rigidly connected with said plate at a fixed distance from one end thereof, oriented at a fixed angle to the plane of said plate, pointing downward and toward said one end, and having an upper edge,

a rigid elongate probe, at least a part of said probe passable longitudinally through said guide tube, and

measuring means to measure the position of said elongate probe longitudinally through said guide tube, relative to said upper edge, and thereby the position of an end of said probe relative to said one end of said plate.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

### BRIEF DESCRIPTION OF THE DRAWINGS

FIG. 1 is a diagrammatic side elevation of a utility pole, in ground, depicting the different testing locations and methods applied to the pole.

FIG. 2 is a cross section of a utility pole including internal anomalies and with an array of acousto-ultrasonic couplings inserted around its circumference.

FIG. 3 is the cross section of FIG. 2 depicting testing along a set of test paths from a coupling device used as a transmitter point.

FIG. 4 is a diagram of a generic circular cross section overlayed with a square grid, and including indications of coupling points, transmission paths between coupling points that are tested in a full test pattern and in accordance with the present invention and showing a set of defined regions of the grid, each region characterised by an intersection of transmission parts.

FIG. 5 is an example screen shot of a hand held computer displaying analysed results in accordance with the present invention.

FIG. 6 is a diagrammatic side elevation of a utility pole, in ground, depicting the test apparatus and method for performing a below ground penetration test according to a preferred embodiment of the present invention.

FIG. 7 is a block diagram of the analysis software according to a preferred embodiment of the present invention.

FIG. 8 is a block diagram of a part of the analysis software for making path anomaly determinations according to an alternative embodiment of the present invention.

FIG. 9 is a block diagram of a part of the analysis software for assisting a user to make path anomaly determinations according to an further alternative embodiment of the present invention.

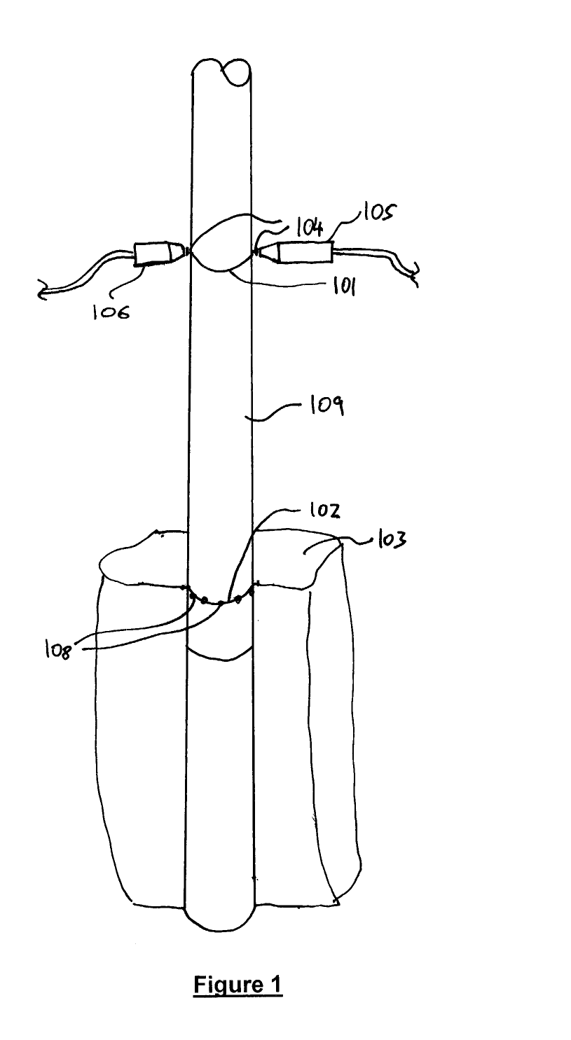

### BRIEF DESCRIPTION OF THE APPENDICES

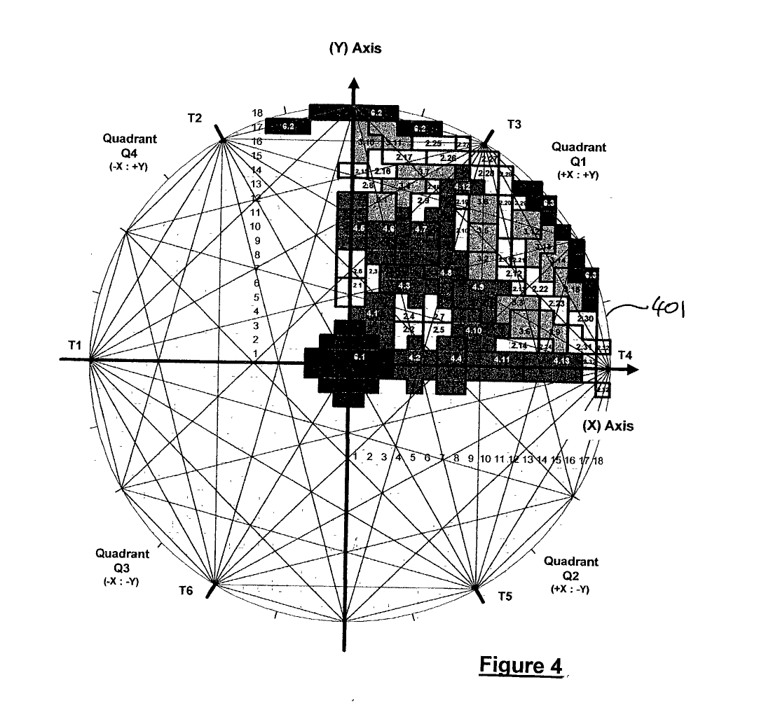

Appendix A is look-up table including data for correlating no-go transmission result combinations with suspect regions of a unit cross section of FIG. 4, and with corresponding discounts for unit second moment of areas about the X and Y axis.

Appendix B is a look up table including data correlating wood classifications with nominal Pilodyn penetrations and a design strengths for each wood classification.

### DETAILED DESCRIPTION

The complete testing system for wooden structural members according to the preferred embodiment of the present invention takes information gathered using both known and new tools, and with improved information gathering methodology, and analyses the information to produce a quantitative measure of the residual strength of the wooden structural member. This system is particularly designed for use with ground embedded poles, such as utility poles or structural support poles, however elements of the system may be used independently to assess internal degradation of other wooden structural members.

Briefly the testing methodology applied to a ground embedded wooden structural member in accordance with the preferred embodiment of the present invention comprises the following steps:

1. A Pilodyn tester is fired into sound wood at a height approximately two metres above ground line at a minimum of three locations around the pole and the average penetration reading is recorded. These readings should be representative of sound wood and should not be taken over obvious defects such as pockets of soft rot or cracks. The Pilodyn instrument fires a spring loaded striker pin into the wood sample and produces a measurement of the depth of penetration. The injection energy from the striking action and dimensions of the pin are both fixed. Therefore there is a correlation between the depth of penetration and the crushing strength of the timber sample. The Pilodyn measurement is recorded in a data input form of a handheld computer and is subsequently used in a residual strength analysis look up to provide additional resolution in a residual bending strength calculation.

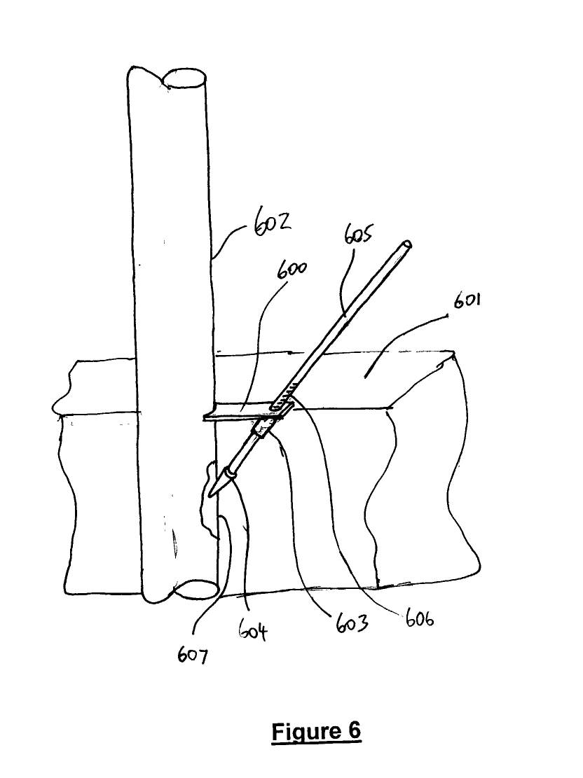

2. A probe tool is used to measure the depth of the degradation of the timber at a depth of approximately 300 mm below ground line. With reference to FIG. 6 the probe tool includes a guide plate 600 placed flat on the ground 601 and butting the base of the pole 602 to be held in place under the user's foot. The guide plate 600 includes a guide tube 603 angled at 35° from the vertical and directed towards the pole 602. A steel shafted probe 604 passes through the guide tube and can be hammered at its above ground end. The probe shaft 605 is calibrated with adjusted depth readings 6069. With one foot on the guide plate a hammer is used to drive the probe through the guide hole until it is met with a firm resistance where successive blows of the hammer do not produce a notable change in penetration depth. The depth of penetration into the pole surface 607 is read off the scale 606 marked on the rod 605 at the level of the top surface of the guide plate 600. The guide plate 600 sets the rod at an angle of 35° from the vertical and at a set distance from the pole at ground level. The scale on the rod correlates to a direct measure of the reduction in pole radius from the original dimension. This procedure is repeated at a minimum of three locations around the pole and the average penetration reading is recorded in the data input form of the handheld computer.

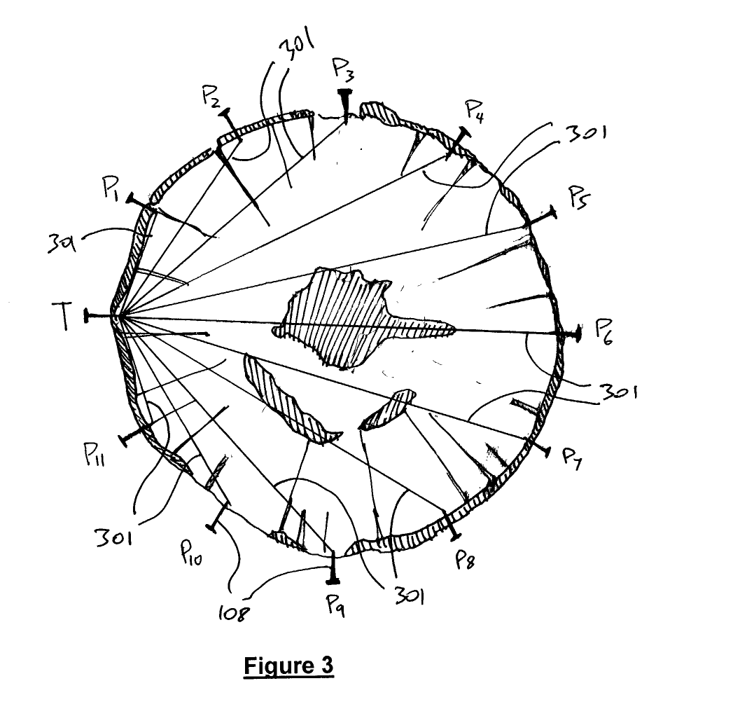

3. Referring to FIG. 3 an acousto-ultrasonic scanner is used to take a series of test readings across various chords of the pole cross section 102 at ground level 103. In a calibrating procedure a first test reading is taken at a level 101 approximately two metres above ground line. This involves driving clout nails 104 into the pole to a depth of 5-10 mm greater than the maximum Pilodyn penetration depth (from (1) above) spaced diametrically across the pole section. The pole diameter is measured using a calibrated diameter tape or callipers. The diameter is recorded in the data input form of the hand held computer as the datum diameter. A sonic pulse propagation time between clouts 104 is measured using an ascousto-ultrasonic testing tool including a broad spectrum transmitter probe 105 held against one nail head and a receiver probe 106 held against the other nail head. Wood at this level is presumed sound and this sonic pulse propagation time represents a presumed propagation velocity for sound timber of the pole type being tested. To accord for insignificant variations in timber density, a nominal additional time (eg: 20 μs) maybe added to the datum propagation time for use for subsequent steps. The transmission time is recorded as the datum time.

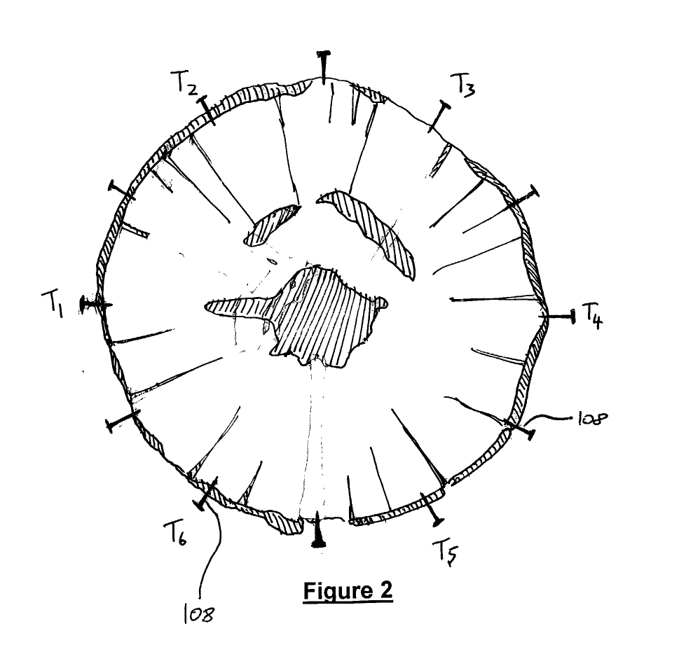

4. An initial scan is taken of the timber cross section 102 (approximately 50 mm above ground line for convenience). With reference to FIG. 2 twelve clouts 108 are driven into the pole 109 to a depth 5-10 mm greater than the Pilodyn penetration reading. The nails 108 are equally spaced around the circumference of the pole (30° apart). The pole diameter is measured using the diameter tape or callipers and recorded on the handheld computer data capture form. An adjusted sound wood threshold transmission time is calculated for the ground level cross-section by compensating for the difference in diameters. The threshold is displayed. Acousto-ultrasound propagation time readings are taken on diametric paths between at least three evenly spaced opposite nail pairs. For example pairs (T₁, T₄), (T₂, T₅) and (T₃, T₆) in FIG. 2. These readings are compared with the threshold time. If all three readings are less than or equal to the predicted time this indicates a lack of internal anomalies and no further scans are necessary for the pole being tested. The scan results are recorded in the data capture form and testing can commence on the next pole.

5. If any of the three diametric readings is greater than the calculated threshold then a further test set of acousto-ultrasonic propagation time readings is completed. Referring to FIGS. 2 and 3, for each transmitter location T1 to T6 a set of 11 chords (eg: chords 301) are tested. The 11 chord testing pattern used for each transmitter position is depicted in FIG. 3. The test results are recorded proceeding in a clockwise direction from the transmitter nail to produce a set of readings P1 to P11 for each transmitter location.

6. Each reading is converted to either a “go” or “no-go” result, a no-go result indicating a presence of an anomaly in the path of the tested chord 301. In converting readings to a go or no-go result the threshold is first adjusted for each reading location to be a specified fraction of the diametric threshold. The fractions represent the shortened path lengths of the non-diametric chords 301. For test readings P1 to P11 these fractions are 0.26, 0.50, 0.70, 0.86, 0.99, 1.00, 0.99, 0.86, 0.70, 0.50 and 0.26 respectively. A test reading along a chord 301 which exceeds the respective fraction of the threshold time is recorded as a no-go result, indicating the presence of an anomaly in the chord path. The readings are recorded on the hand held computer data capture form as a 6×11 array of results.

7. The 6×11 array of results is processed through a look-up table by software executed on the computer. Specified combinations of no-go results are presumed to indicate suspect pre-defined regions of the tested cross section 102. An example pattern 401 of pre-defined regions is shown in FIG. 4. The regions shown in FIG. 4 make a first quadrant Q1 of a circular cross section. The defined areas shown in the first quadrant apply equally to the other quadrants (Q2 to Q3) through symmetry about the X-axis and the Y-axis. The required no-go combinations are specified for each respective quadrant Q1 to Q4 in the look-up table. A corresponding look-up table is included as Appendix A. The example look up table includes “region ID” in a first column, corresponding required no-go results for the corresponding region of each quadrant in a second column, a perimeter path definition for the region in a third column, a total region size (in unit squares) in the fourth column, a tabulation of unit distances from the respective X and Y axii in a fifth and sixth column respectively and unit second moment of area subtraction values (with respect to the xx-axis and yy-axis respectively) in seventh and eighth columns.

8. The executed software calculates residual strength characteristics for the structural member by subtracting unit second moment of area reductions of suspect regions from the total unit second moment of area of an assumed sound test section, and by calculating an absolute second moment of area about both the X and Y axii from the unit second moments of area and a recorded section diameter. The calculated absolute second moment of area for the below ground cross section assumes that internal anomalous structures correspond with internal anomalies at ground level (ie: the unit second moment of area of the below ground cross section is the same as that of the ground level test section but the radius is reduced by the measured surface rot depth).

9. A wood strength classification is selected on the handheld computer corresponding to the wood type of the pole being tested. From a lookup table the software finds an ultimate limit state Modulus of Rupture MoR (in MPa) corresponding to the Pilodyn datum penetration. An example lookup table is shown in Appendix B. The table in Appendix B includes standards based wood clarifications and design strengths. The Pilodyn datum penetrations are an average penetration for tests over a range of timbers in the class and in varying age condition. The Pilodyn datum is used to account for significant variation in timber properties within each wood classification. For example the Mixed Australian Hardwood (MAH) class includes Ironbark, extremely strong and durable, and Blackbutt, which barely makes the necessary grade. The Pilodyn datum is also used to compensate for surface degradation of the pole. The Pilodyn datum and the Pilodyn penetration reading are used to calculate a modified section diameter for use in recalculating the absolute second moments of area used for calculating the bending strength. In particular the modified diameter is found from:

d=(2 m−2 P )+D

where D is the measured girth for the cross section in question (if below ground then D is further adjusted for the surface rot measurement), m is the Pilodyn datum from the lookup table, P is the Pilodyn depth measurement (from (1) above) and d is the modified diameter. Modified residual second moments of area are calculated from the modified diameter and the residual unit second moments of area.

The Pilodyn penetration can also be used to select a wood strength classification, by administering the Pilodyn test on sound timber and making a determination a determination of the wood strength classification by comparing the penetration depth with the penetration depths on the look up table.

10. The computer software calculates an overall ultimate limit state bending load for the ground level cross section and below ground cross section from the modified residual second moments of area and the timber strength listed in the look up table for the relevant wood class.

The software executed on the handheld computer may receive test results directly from the acousto-ultrasonic testing probes or alternatively the data may be keyed by a user. Where the data is keyed in by a user the go/no-go evaluation may be made by the user or the raw test result may be entered and the go/no-go evaluation made by a software executed comparison.

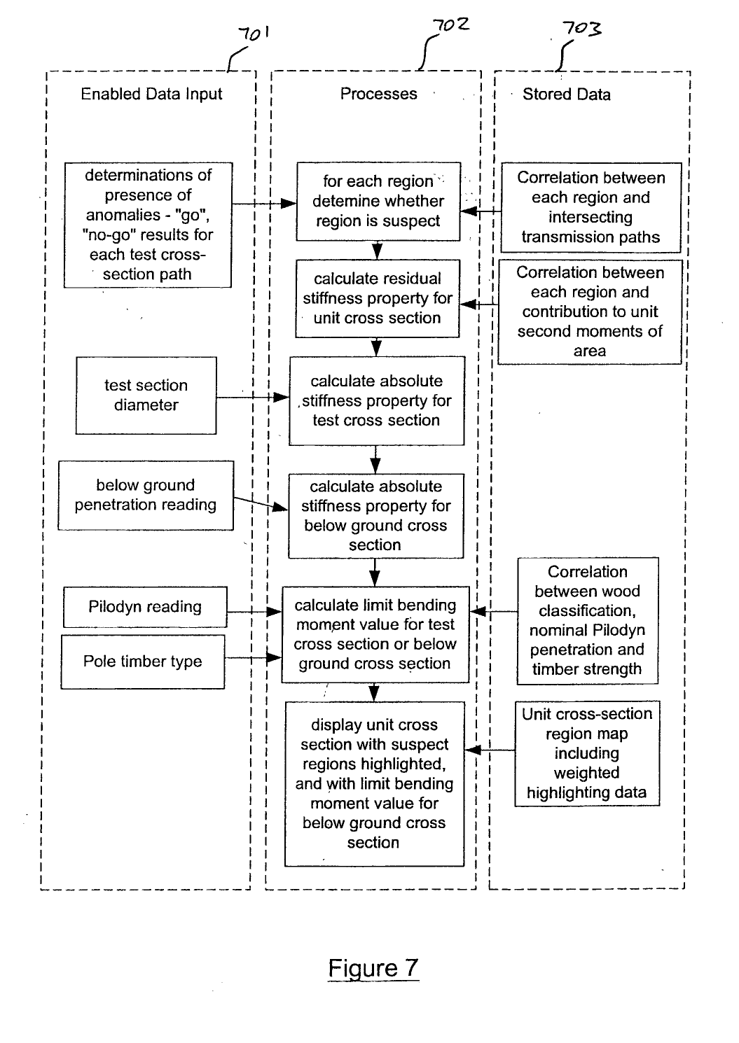

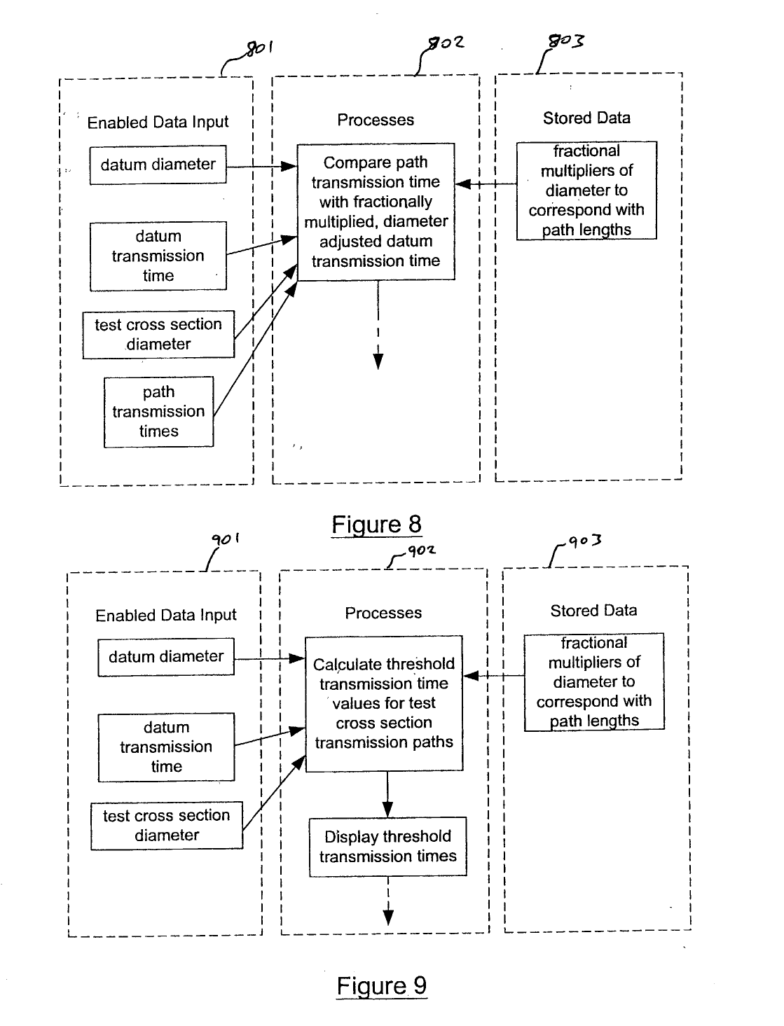

FIGS. 7-8 are block diagrams representing the computer software process executed on the handheld computer. In particular FIG. 7 shows a system in which a user makes the go/no-go evaluations without automated assistance and enters these directly to the handheld computer. FIG. 8 represents a variation to this system in which the go/no-go determine is made by the software on the basis on received data. FIG. 9 relates to a further variation in which the computer software provides assistance to the user to make the go/no-go determination by displaying a set of threshold values.

In the block diagram of FIG. 7 the enabled data inputs are enclosed by box 701 and that data pre-stored on the handheld computer is enclosed by box 703. The sequential process that is executed is enclosed by box 702 and the links between particular data inputs or stored data and individual process steps indicate the reliance of that process step on the respective input data or stored data. The executed process of FIG. 7 is described earlier apart from the final display step which is described below.

Referring to FIG. 8, the go/no-go determination maybe made by the software on the basis of received data indicating the datum diameter and datum transmission time, the test cross section diameter and the transmission times for the test cross section paths. In common with FIG. 7, in FIG. 8 enabled data inputs are enclosed by box 801 and data pre-stored in the computer is enclosed by box 803. The additional process step enclosed by box 802 draws on the data inputs in box 801 and the stored data in box 803. The datum transmission time is multiplied by the ratio (test cross section diameter)/(datum diameter) and the result is multiplied by the fractional multipliers to produce a set of threshold transmission times for the test cross section paths. The software compares the actual transmission times (received either by user input or determined based on readings received directly from the probes or from an electronic interface), with the threshold times and determines a no-go result for a path if the actual transmission time exceeds the corresponding threshold time.

Referring to FIG. 9 where the go/no-go determination is left to a user the software may still provide assistance. In common with FIG. 7, in FIG. 9 enabled data inputs are enclosed by box 901 and data pre-stored in the computer is enclosed by box 903. The additional process step enclosed by box 902 draws on the data inputs in box 901 and the stored data in box 903. In particular the software may calculate a set of threshold transmission time values for no-go readings for the test cross-section transmission paths. These may be calculated from the datum diameter, the datum transmission time, the tests section diameter and the stored set of fractional multipliers. The software may display the set of comparison values to the user for the convenience of the user.

Where the readings are transmitted directly to the handheld computer these may be conducted from the transmitting and receiving probes via cables with a translating interface or the raw timing information may be wirelessly transmitted to a receiver interfaced with the handheld computer for further processing by the handheld computer.

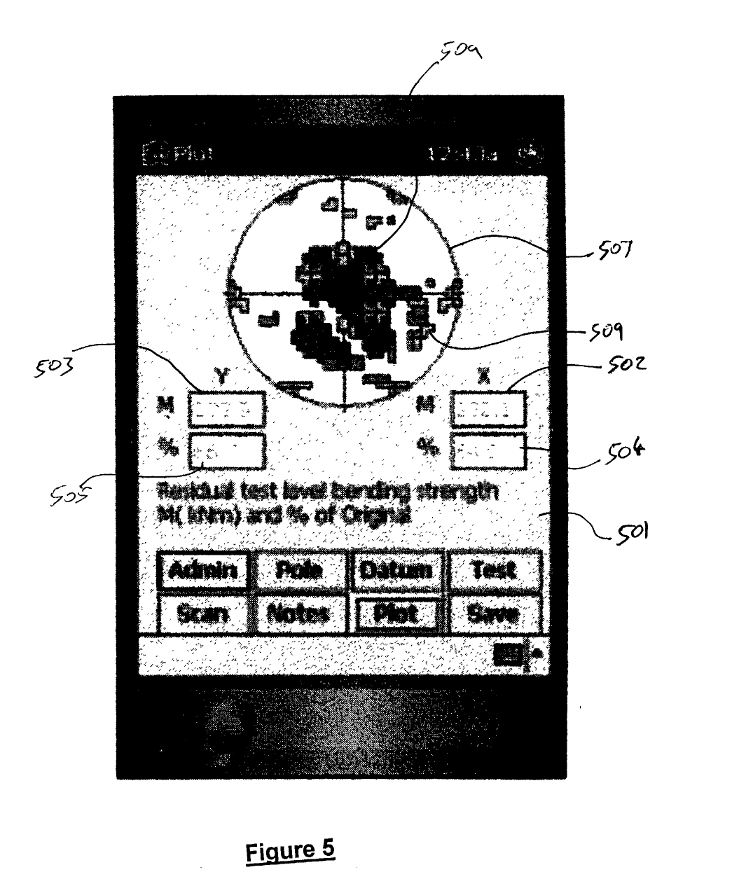

In addition to calculating and recording the residual pole strength the handheld computer is programmed to display the results to a user in simplified format. An example display screen shot 501 showing test results is included as FIG. 5. In this example the residual safe bending strength 502, 503 about both the Y and X (respectively) axes are displayed together with an indication of these strengths as a percentage 504, 505 of the presumed original strength (ie: calculated as if having no internal anomalies). In addition a display 507 of the pole unit cross section highlights the suspect regions identified at step 7 above. The suspect regions (regions eg 509) are highlighted so as to differentiate between the suspect regions depending on the likely reliability of the result. In particular, regions identified as suspect by the intersection of more no-go readings are taken to have a higher likelihood of being suspect than regions identified as suspect by the intersection of fewer no-go readings. The appropriate highlighted for each region is indicated by the shading of that region in FIG. 4.

In the system of the present invention determination of a region as suspect may result from shadowing. That is, one or more of the test results that contribute to determination of that region as suspect may have been caused by anomalies in other regions along its path.

At this stage this basic probability analysis is only used to provide a visual indication. So far as the residual strength calculation is concerned any region identified as suspect has its unit component of the second moment of area discounted. A more complex calculation could be made weighting these discounts according to the perceived probability of the suspect region determination proving accurate. Furthermore it is possible to determine whether suspicion of a region could actually result from shadowing in a given tested cross section by reviewing the determinations for other regions. For example, a region may be suspect due to shadowing if each of the no-go determinations that cause it to be suspect is also involved in causing another region to be suspect. Where one or more regions of a group of regions determined as suspect could be the result of shadowing the software could be adapted to not discount those regions of lowest probability from the second moment of area calculations. In the case of any such non-discounting the software should make a residual check to ensure that every “no-go” reading still has an associated region regarded as suspect for which a discount is being applied.

However at present such embellishments to the software have not been found necessary for the production of suitably accurate results. In tests on a population of 342 poles the system has produced accurate plots of the pole cross sections showing the locations and extent of significant internal anomalies. The output calculations of residual second moments of area have mean value accuracies ranging from 88% to 92% with scatter coefficients of variation ranging from 0.11 to 0.15. The system operating as described provides an invariably conservative mapping of internal anomalies, assuming some areas to be anomalous which may well prove otherwise. However the two dimensional location of suspect regions on a pre-determined array of regions allows calculation of a reasonably accurate second moment of area while maintaining the viability of the system as a field testing procedure. In testing a sample population of 335 poles the average time taken for a trained person to conduct a full and complete analysis at one cross section was found to be less than 10 minutes.

The preferred embodiment of the present invention has been described with particular reference to the testing of utility poles and other ground embedded poles. However it will be appreciated that most parts of the present system may also be used for testing other wooden structural members and in addition for the testing of trees, whether living or dead, and logs.

Advantages accruing from the present system include a quantitative assessment of residual pole strength and a visual guide to internal structural integrity within a system well adapted to the rigours of field application.

[image]

[image]

[image]

[image]

[image]

[image]

[image]

1. A method of testing a wooden structural member to provide a quantitative estimate of the residual strength thereof comprising, in any order:
determining the presence of anomalies in each of a plurality of intersecting transverse paths through said structural member at a cross section of said structural member,
for each region of a plurality of regions, said plurality of regions together making up a unit cross section of a structural member of the shape being tested and each region including a point of intersection of at least two said paths, determining the region as suspect if each of the paths intersecting at said region has indicated an anomaly, and
calculating a residual stiffness property for said cross section, discounting suspect regions.

2. A method of testing a wooden structural member as claimed in claim 1 including the steps of measuring a cross sectional dimension of said structural member and calculating a quantitative estimate of the residual absolute second moment of area from said calculated stiffness property for the unit cross section and from said measured cross sectional dimension.

3. A method of testing a wooden structural member as claimed in claim 2 wherein measuring said cross sectional dimension includes:
making a dimensional measurement of the structural member at an above ground location, and
probing the structural member to a surface depth indicating sound wood at a below ground location, from a known position laterally displaced from the surface of said structural member at ground level and with the probe at a known angle to the longitudinal direction of said structural member, said depth of insertion of said probe indicating the reduction in dimension at a known below ground level compared with the dimension at ground level.

4. A method of testing a wooden structural member as claimed in any one of claims 1 to 3 including the steps of determining a residual limit strength of sound material of said member and calculating a quantitative estimate of the residual limit bending moment for the structural member from said determined residual limit strength and said calculated absolute second moment of area.

5. A method of testing a wooden structural member as claimed in claim 4 wherein determining a residual limit strength of sound material of said member includes executing a known energy impact penetration test on apparently sound material of said member, and comparing the depth of penetration from said test with a known correlation in a lookup table.

6. A method of testing a wooden structural member as claimed in claim 4 including the step of displaying one or more of said calculated unit second moment of area, said absolute second moment of area and said absolute limit bending moment.

7. A method of testing a wooden structural member as claimed in any one of claims 1 to 7 including the step of displaying a visual representation of said cross section of said wooden structural member with said suspect regions highlighted.

8. A method of testing a wooden structural member as claimed in claim 7 wherein in said step of displaying visual representation, said regions are highlighted in accordance with a schema wherein the highlighting of suspect regions is differentiated in accordance with the number of paths that intersect at the said region.

9. A method for testing for internal anomalies in a wooden structural member comprising the steps of:
determining the presence of anomalies in each of a plurality of intersecting transverse paths through said structural member at a cross section of said structural member,
for each region of a plurality of regions, said plurality of regions together making up a unit cross section of a structural member of the shape being tested and each region including a point of intersection of at least two said paths, determining the region as suspect if each of the paths intersecting at said region has indicated an anomaly, and
displaying a visual representation of said cross section of said wooden structural member with suspect regions highlighted.

10. A method of testing for internal anomalies in a wooden structural member as claimed in claim 1 wherein in said step of displaying a visual representation, said regions are highlighted in accordance with a schema wherein the highlighting of suspect regions is differentiated in accordance with the number of paths that intercept at the said region.

11. Apparatus for use in testing a wooden structural member to provide a quantitative estimate of the residual strength thereof comprising:
means for receiving a determination of the presence of anomalies in each of a plurality of intersecting transverse paths through said structural member at a test cross section of said structural member,
processing means for determining for each region of a plurality of regions, said plurality of regions together making up a unit cross section of a structural member of the shape being tested and each region including a point of intersection of at least two said paths, the region as suspect if each of the paths intersecting at said region has indicated an anomaly, and
processing means for calculating a residual stiffness property for said unit cross section, discounting suspect regions.

12. Apparatus for use in testing a wooden structural member as claimed in claim 11 including means for receiving at least one reading representative of a transmission velocity for an acousto-ultrasonic transmission across a diameter of said structural member deemed sound.

13. Apparatus for use in testing a wooden structural member as claimed either claim 11 or claim 12 including means for receiving readings representative of transmission velocities for acousto-ultrasonic transmissions along said paths of said test cross section and means for determining the presence of anomalies on a said path by directly or indirectly comparing the transmission velocity for said path with said velocity for material deemed sound.

14. Apparatus for use in testing a wooden structural member as claimed in claim 12 wherein said representative readings are transmission times for acousto-ultrasonic transmissions along the respective paths and said apparatus includes means to receive a measurement of a diameter of said presumed sound cross-section, and means to calculate a datum transmission time for transmissions across said test cross-section.

15. Apparatus for use in testing a wooden structural member as claimed in claim 14 wherein including means to determine the presence of anomalies along a said path of said test-cross section by comparing said transmission times along said test cross-section paths with fractional multiples of said datum transmission time.

16. Apparatus for use in testing a wooden structural member as claimed in claim 14 including means to display fractional multiples of said datum transmission time adjusted for the expected reduce path length of said oaths relative to a diameter, for a user to make a determination.

17. Apparatus for use in testing a wooden structural member as claimed in any one of claims 11 to 16 including means for receiving a measurement of a cross sectional dimension of said structural member and processing means for calculating a quantitative estimate of the residual absolute second moment of area from said calculated stiffness property for the unit cross section and from said measured cross sectional dimension.

18. Apparatus for use in testing a wooden structural member as claimed in claim 17 including means for receiving a determined residual limit strength of sound material of said member and processing means for calculating a quantitative estimate of the residual limit bending moment for the structural member from said determined residual limit strength and said calculated absolute second moment of area.

18. Apparatus for use in testing a wooden structural member as claimed in claim 18 including said apparatus includes means for receiving the penetration depth result of a known energy impact penetration test and means for making a determination of a residual limit strength of sound material of said member, said means comparing the depth of penetration from said test with a known correlation.

20. Apparatus for use in testing a wooden structural member as claimed in either claim 18 or claim 19 including said apparatus includes a display and means for displaying one or more of said calculated unit second moment of area, said absolute second moment of area and said absolute limit bending moment on said display.

21. Apparatus for use in testing a wooden structural member as claimed in claim any one of claims 11 to 20 including means for displaying a visual representation of said cross section of said wooden structural member with said suspect regions highlighted.

22. Apparatus for use in testing a wooden structural member as claimed in claim 21 wherein said means for displaying visual representations displays said regions highlighted in accordance with a schema wherein the highlighting of suspect regions is differentiated in accordance with the number of paths that intercept at the said region.

23. Apparatus for use in testing for internal anomalies in a wooden structural member comprising:
means for receiving a determination of the presence of anomalies in each of a plurality of intersecting transverse paths through said structural member at a cross section of said structural member,
processing means for determining for each region of a plurality of regions, said plurality of regions together making up a unit cross section of a structural member of the shape being tested and each region including a point of intersection of at least two said paths, the region as suspect if each of the paths intersecting at said region has indicated an anomaly,
a display, and
means for displaying a visual representation of said cross section of said wooden structural member with suspect regions highlighted.

24. Apparatus for use in testing for internal anomalies in a wooden structural member as claimed in claim 23 wherein said means for displaying visual representations displays said regions highlighted in accordance with a schema wherein the highlighting of suspect regions is differentiated in accordance with the number of paths that intercept at the said region.

25. A below ground penetration test tool comprising:
a plate,
a guide tube rigidly connected with said plate at a fixed distance from one end thereof, oriented at a fixed angle to the plane of said plate, pointing downward and toward said one end, and having an upper edge,
a rigid elongate probe, at least a part of said probe passable longitudinally through said guide tube, and
measuring means to measure the position of said elongate probe longitudinally through said guide tube, relative to said upper edge, and thereby the position of an end of said probe relative to said one end of said plate.

26. A below ground penetration test tool as claimed in claim 25 wherein said measuring means includes a graduated scale marked on said part of said probe extending through said guide tube, and said relative position of said probe end can be read directly off said graduated scale at said upper edge.
